(19) United States
(12) Patent Application Publication
(10) Pub. No.: US 2005/0261499 A1
(43) Pub. Date: Nov. 24, 2005

(54) Process for preparing (r)-aryloxypropionic acid ester derivatives

(76) Inventors: Dae Whang Kim, Daejeon (KR); Kun Hoe Chung, Daejeon (KR); Hae Sung Chang, Daejeon (KR); Young Kwan Ko, Daejeon (KR); Jae Wook Ryu, Daejeon (KR); Jae Chun Woo, Daejeon (KR); Dong Wan Koo, Daejeon (KR)

Correspondence Address: Ronald R Santucci;Frommer Lawrence & Haug, 745 Fifth Avenue, New York, NY, 10151, US

(21) Appl. No.: 10/518,566

(22) PCT Filed: Jun. 25, 2003
(86) PCT No.: PCT/KR03/01244

§ 371 Date: Dec. 20, 2004

(30) Foreign Application Priority Data
Jun. 26, 2002 (KR) 10-2002-0036051

(51) Int. Cl. C07C 69/76; C07D 277/62; C07D 213/63

(52) U.S. Cl. 544/333; 548/157; 548/216; 560/55; 560/56; 558/410; 546/290

(57) ABSTRACT

The present invention relates to a method for preparing optically active (R)-aryloxypropionic acid ester derivatives, and more particularly to a method for preparing (R)-aryloxypropionic acid ester derivatives with high optical purity and good yield at low cost from phenol derivatives with various substituted functional groups and (S)-alkyl O-arylsulfonyl lactates.

### TECHNICAL FIELD

The present invention relates to a method for preparing optically active (R)-aryloxypropionic acid ester derivatives, and more particularly to a method for preparing (R)-aryloxypropionic acid ester derivatives represented by the following formula 1 with high optical purity and good yields at low cost via nulceophilic substitution reaction using phenol derivatives with various substituted functional groups and (S)-alkyl O-arylsulfonyl lactates as reactants in the presence of a proper solvent and a base at optimum temperature: 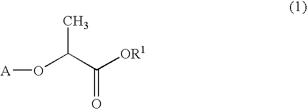
wherein R¹ is a C₁₋₆-alkyl or benzyl group; A is an aryl group selected from the group consisting of a phenyl group, a naphthyl group, quinoxazolyloxyphenly group, a benzoxazolyloxyphenyl group, a benzothiazolyloxyphenyl group, a phenoxyphenol group, a pyridyloxyphenyl group and a phenyloxynaphthyl group, wherein the aryl group can be substituted with 1-3 functional groups selected from the group consisting of a hydrogen atom, a halogen atom, a nitro group, a nitrile group, an acetoxy group, a C₁₋₄-alkyl group, a C₁₋₄-haloalkyl group, a C₁₋₄-alkoxy group, and a C₁₋₄-haloalkoxy group.

### BACKGROUND ART

The compound represented by Formula 1, commonly called (R)-propionic acid ester, is well known as a herbicidal substance that inhibits physiological functions of plants. Among them, a few compounds including (R)-ethyl 2-[4-(6-chloro-2-benzoxazolyloxy)phenoxy]propionate have been used as agrochemicals.

Due to the presence of a single chiral carbon, the 2-substituted propionic acid ester derivatives as represented above have optical isomers. In particular, it is known that their (R)-isomers have herbicidal activities while their (S)-isomers are of little herbicidal activities.

Preparation of propionic acid derivatives and their herbicidal activities have been disclosed in literatures [European Patent Nos. 157,225, 62,905, and 44,497; German Patent Nos. 3,409,201, 3,236,730, and 2,640,730].

The conventional methods of preparing propionic acid derivatives are well represented by the following two reaction schemes 1 and 2.

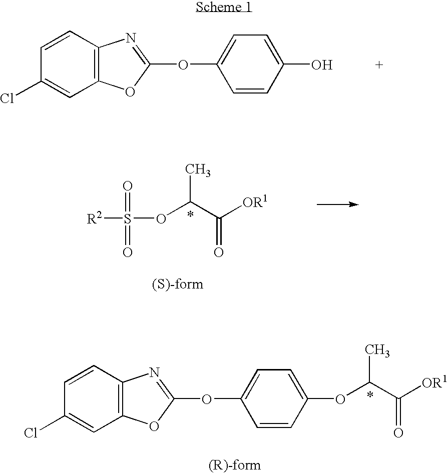

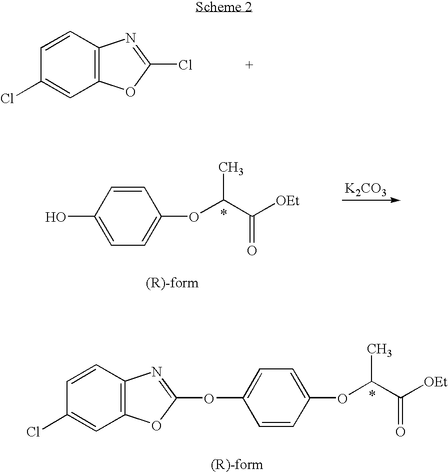

In the above methods of scheme 1, wherein substituted phenol and (S)-alkyl O-sulfonyl lactate are reacted, and scheme 2, wherein 2,6-dichlorobenzoxazole and (R)-ethyl 2-(4-hydroxyphenoxy)propionate are reacted, the reactions are performed in a polar solvent including acetonitrile to obtain (R)-fenoxaprop ethyl [yield=70-80%; optical purity=60-90%].

However, these methods generate about 5-20% of (S)-isomers as by-products, which are not easily removed, and thus a rather complex process such as recrystallization is required to obtain pure (R)-fenoxaprop ethyl, thus increasing cost in preparation. Further, it is also a burden that starting materials, (R)-alkyl 2-(4-hydroxyphenoxy)propionates used in the reactions are to maintain high optical activity.

The inventors of the present invention focused on developing a novel method for preparing (R)-propionic acid ester derivatives, which have high optical purity with good yield. In doing so, the inventors of the present invention realized that it is important to find an appropriate condition for nucleophilic substitution reaction that prevents racemization of propionic acid ester derivatives. Accordingly, an object of the present invention is to provide a novel method for preparing optically active (R)-propionic acid ester derivatives at low cost by preventing racemization.

### DISCLOSURE OF INVENTION

The present invention relates to a method for preparing (R)-propionic acid ester derivatives with high optical purity by reacting phenol derivatives represented by the following Formula 2 and (S)-alkyl O-arylsulfonyl lactate represented by the following Formula 3 in the presence of alkali metal carbonate base in an aliphatic or aromatic hydrocarbon solvent at 60-100° C: 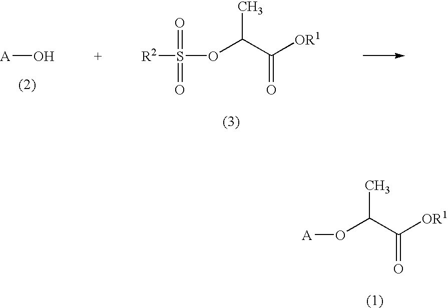
wherein R¹ is a C₁₋₆-alkyl or benzyl group; R² is a C₁₋₆-alkyl, phenyl group, or a phenyl group substituted with a C₁₋₆-alkyl or a C₁₋₆-alkoxy group; A is an aryl group selected from the group consisting of a phenyl group, a naphthyl group, a quinoxazolyloxyphenly group, a benzoxazolyloxyphenyl group, a benzothiazolyloxyphenyl group, a phenoxyphenol group, a pyridyloxyphenyl group and a pheyloxynaphthyl group, wherein said aryl group can be substituted with 1-3 functional groups selected from the group consisting of a hydrogen atom, a halogen atom, a nitro group, a nitrile group, an acetoxy group, a C₁₋₄-alkyl group, a C₁₋₄-haloalkyl group, a C₁₋₄-alkoxy group, and a C₁₋₄-haloalkoxy group.

Hereinafter, the present invention is described in more detail.

The present invention relates to a method for preparation of optically active (R)-propionic acid ester derivatives with high yield and good optical purity via nucleophilic substitution reaction using phenol derivatives and (S)-alkyl O-arylsulfonyl lactates as reactants, wherein the reactions are performed under a condition of solvent, temperature and leaving group, which are all specifically designed.

Phenol derivatives and (S)-alkyl O-arylsulfonyl lactates, reactants of the present invention as represented by the above Formulas 2 and 3, are known compounds and are synthesized by the known methods. For example, (6-chloro-2-benzoxazolyloxy)phenol can be prepared by a 4-step reaction using commercially available substances, such as aminophenol, urea, sulfuryl chloride, phosphorus pentachloride, and triethylamine, and solvents, such as xylene, acetic acid, chlorobenzene, and dichloroethane. And, (S)-alkyl O-arylsulfonyl lactate can be prepared by reacting (S)-alkyl lactate and arylsulfonyl chloride in the presence of triethylamine in dichloroethane solvent.

In the nucleophilic substitution reaction of the present invention, selection of the reaction solvent plays a crucial role in preventing racemization. As a reaction solvent, aliphatic or aromatic hydrocarbon solvents such as xylene, toluene, benzene, cyclohexane, methylcycloheane, n-hexane, and n-heptane, etc. can be used, and cyclohexane and xylene are preferred among them.

The reaction temperature is also a very important factor to prevent racemization. A temperature range of 60-100°C. is appropriate, but considering reaction time and convenience, reflux temperature of cyclohexane (˜80° C.) is particularly preferable.

As a base of the present invention, alkali metal carbonates such as sodium carbonate, potassium carbonate, etc., can be used. Production of metal salt of phenol as an intermediate using the alkali metal carbonate as a base can greatly reduce unnecessary side reactions. Further, the above base is preferred to be powder (400-700 mesh) rather than pellets because powder form can reduce reaction time.

In the nucleophilic substitution reaction according to the present invention, water is generated as a byproduct while phenol-metal salt is produced as a main reaction intermediate. Thus generated water is removed by use of a specifically selected solvent in the present invention and this leads to a more effective prevention of racemization of products as well as hydrolysis of ester.

Upon completion of the nucleophilic substitution reaction, the sulfonic acid salt is filtered without cooling, and the filtrate is condensed to obtain (R)-propionic acid ester derivatives represented by Formula 1, the target compound of the present invention with high yields and good optical purity.

This invention is further illustrated by the following examples, however, these examples should not be construed as limiting the scope of this invention in any manner.

### BEST MODE FOR CARRYING OUT THE INVENTION

### EXAMPLE 1

### Preparation of (D+)-ethyl-2-(4-chloro-2-methylphenoxy)propionate (Compound 1)

30 mL of cyclohexane, 1.43 g (10 mmol) of 4-chloro-2-methylphenol, 2.86 g (10.5 mmol) of (S)-ethyl O-p-toluenesulfonyl lactate, and 2.76 g (20 mmol) of powdery K₂CO₃ were put in a 50 mL flask equipped with a cooling condenser-attached Dean-Stock and reacted for 17 hours while refluxing. The reaction mixture was filtered without cooling and the solid cake was washed with 20 mL of warm cyclohexane. The cyclohexane layer, the filtrate, was condensed to obtain 2.26 g of the target compound (yield=93%; purity=98%; optical purity=99.4%).

Rf=0.68(EA:Hx=1:4); 1H NMR(CDCl3, 200 MHz) δ 1.24(t, J=7.2 Hz, 3H), 1.62(d, J=6.8 Hz, 3H), 2.25(s, 3H), 4.20(q, J=7.2 Hz, 2H), 4.69(q, J=6.8 Hz, 1H), 6.58^{˜}7.13(m, 3H); MS(70 eV) m/z 244(M+), 242(M+), 169, 142, 125, 107, 89, 77

The following Table 1 shows the yield, ratio of generated optical isomers and spectral data of the compounds (1-25) performed the same as in Example 1.

TABLE 1

| comp. | | R/S | | |
|---|---|---|---|---|
| no. | structure | ratio | yields | mp, R_{f}, NMR, MS |
| 1 | 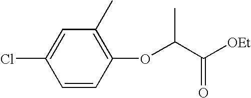 | 99.4/ 0.6 | 93% | yellow liquid; R_{f}=0.68(EA:Hx=1:4); ¹H NMR(CDCl₃, 200MHz) δ1.24(t J=7.2Hz, 3H), 1.62(d, J=6.8Hz, 3H), 2.25(s, 3H), 4.20(q, J=7.2Hz, 2H), 4.69(q, J=6.8Hz, 1H), 6.58 \tilde{ } 7.13(m, 3H); MS(70eV) m/z 244(M⁺), 242(M⁺), 169, 142, 125, 107, 89, 77 |
| 2 | 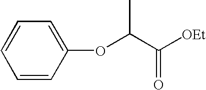 | 83.0/ 17.0 | 70% | white liquid; R_{f}=0.71(EA:Hx=1:3); ¹H NMR(CDCl₃, 200MHz):δ 1.24(t, J=7.1Hz, 3H), 1.62(d, J=6.8Hz, 3H), 4.21(q, J7.2Hz, 2H), 4.74(q, 1=6.8Hz, 1H), 6.93˜7.27(m, 5H); MS(70eV) m/z 194(M⁺), 121, 94, 77,58,43 |
| 3 | 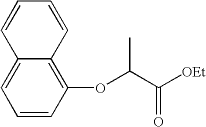 | 86.3/ 13.7 | 76% | yellow liquid; R_{f}=0.70(EA:Hx=1:4); ¹NMR(CDCl₃, 200MHz):δ 1.22(t, J=7.2Hz, 3H), 1.75(d, J=6.8Hz, 3H) 4.21(q, J=7.2Hz, 2H), 4.92(q, J=6.8Hz, 1H), 6.67˜8.38(m, 7H); MS(70eV) m/z 244(M⁺), 199, 171 144, 127, 115, 101, 89 |
| 4 | 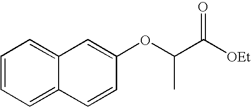 | 88.0/ 12.0 | 82% | yellow liquid; R_{f}=0.63(EA:Hx=1:4); ¹H NMR(CDCl₃, 200 Mz); δ 1.24(t, J=7.1Hz, 3H), 1.68(d, J=6.8Hz, 3H), 4.23(q, J=7.2Hz, 2H), 4.89(q, J=6.8Hz, 1H), 7.04˜7.77(m, 7H); MS(70eV) m/z 244(M⁺), 199, 171, 144, 127, 115, 101, 89 |
| 5 | 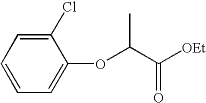 | 100.0/ 0.0 | 97% | yellow liquid; R_{f}=0.67(EA:Hx=1:4); ¹H NMR(CDCl₃, 200MHz): δ 1.25(t, J=7.1Hz, 3H), 1.68(d, J=7.0Hz, 3H), 4.22(q, J=7.2Hz, 2H), 4.75(q, J=6.8Hz, 1H), 6.83˜7.40(m, 4H); MS(70eV) m/z 230(M⁺), 228(M⁺), 193, 194, 155, 128, 111, 99, 91 |
| 6 | 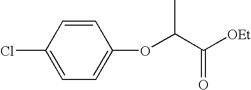 | 84.9/ 15.1 | 98% | yellow liquid; R_{f}=0.70(EA:Hx=1:4); ¹H NMR(CDCl₃, 200MHz): δ 1.25(t, J=7.1Hz, 3H), 1.61(d, 17.0Hz, 3H), 4.21(q, J=7.1Hz, 2H), 4.70(q, J=6.8Hz, 1H), 6.78˜7.25(m, 4H); MS(70eV) m/z 230(M⁺), 228(M⁺), 155, 128, 111, 99, 91, 75 |
| 7 | 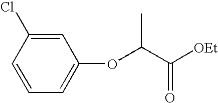 | 97.2/ 2.8 | 96% | yellow liquid, R_{f}=0.65(EA:Hx=1:4); ¹H NMR(CDCl₃, 200MHz): δ 1.26(t, J=7.1Hz, 3H), 1.62(d, J=7.0Hz, 3H), 4.23(q, J=7.2Hz, 2H), 4.72(q, J=6.9Hz, 1H), 6.73˜7.23(m, 4H); MS(70eV) m/z 230(M⁺), 228(M⁺), 155, 128, 111, 99, 91, 75 |
| 8 | 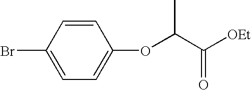 | 96.7/ 3.3 | 96% | white liquid; R_{f}=0.60(EA:Hx=1:4); ¹H NMR(CDCl₃, 200MHz): δ 1.25(t, J=7.1Hz, 3H), 1.61(d, J=7.0Hz, 3H), 4.21(q, J=7.2Hz, 2H), 4.68(q, J=6.8Hz, 1H), 6.74˜7.39(m, 4H); MS(70eV) m/z 272(M⁺), 199, 172, 155, 120, 91 |
| 9 | 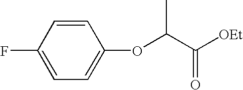 | 94.9/ 5.1 | 95% | white liquid; R_{f}=0.72(EA:Hx=1:4); ¹H NMR(CDCl₃, 200MHz): δ 1.25(t, J=7.1Hz, 3H), 1.60(d, J=7.0Hz, 3H), 4.21(q, J=7.0Hz, 2H), 4.67(q, J=6.8Hz, 1H), 6.79˜7.00(m, 4H); MS(70eV) m/z 212(M⁺), 139, 112, 95, 83 |
| 10 | 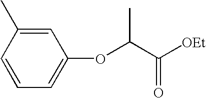 | 93.3/ 6.7 | 98% | white liquid; R_{f}=0.68(EA:Hx=1:4); ¹H NMR(CDCl₃, 200MHz): δ 1.25(t, J=7.1Hz, 3H), 1.60(d, J=7.0Hz, 3H), 2.31(s, 3H), 4.22(q, J=7.2Hz, 2H), 4.73(q, J=6.8Hz, 1H), 6.64˜7.18(m, 4H); MS(70eV) m/z 208(M⁺), 135, 108, 91, 77,65 |
| 11 | 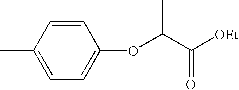 | 94.3/ 5.7 | 94% | white liquid; R_{f}=0.68(EA:Hx=1:4); ¹H NMR(CDCl₃, 200MHz): δ 1.25(t, J=7.2Hz, 3H), 1.60(d, J=6.8Hz, 3H), 2.27(s, 3H), 4.21(q, J=7.2Hz, 2H), 4.70(q, J=6.8Hz, 1H), 6.76˜7.10(m, 4H); MS(70eV) m/z 208(M⁺), 135, 107, 91, 77, 65 |
| 12 | 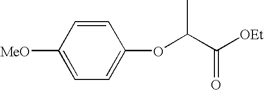 | 95.4/ 4.6 | 88% | white liquid; R_{f}=0.42(EA:Hx=1:4); ¹H NMR(CDCl₃, 300MHz): δ 1.25(t, J=7.1Hz, 3H), 1.59(d, J=6.8Hz, 3H), 3.75(s, 3H), 4.21(q, J=7.1Hz, 2H), 4.65(q, J=6.8Hz, 1H), 6.78˜6.86(m, 4H); MS(70eV) m/z 224(M⁺), 151, 123, 109, 92, 77, 64 |
| 13 | 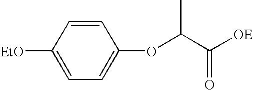 | 98.1/ 2.9 | 82% | white liquid; R_{f}=0.51(EA:Hx=1:4); ¹H NMR(CDCl₃, 300MHz): δ 1.25(t, J=7.2Hz, 3H), 1.38(t, J=7.1Hz, 3H), 1.59(d, J=6.9Hz, 3H), 3.96(q, J=6.9Hz, 2H), 4.21(q, 17.2Hz, 2H), 4.80(q, J=6.8Hz, 1H), 6.78˜6.84(m, 4H); MS(70eV) m/z 238(M⁺), 165, 137, 109, 91, 81, 65 |
| 14 | 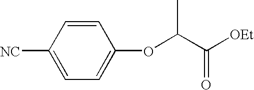 | 100.0/ 0.0 | 100% | white liquid; R_{f}=0.48(EA:Hx=1:2); ¹H NMR(CDCl₃, 300MHz): δ 1.26(t, J=7.2Hz, 3H), 1.65(d, J=6.6Hz, 3H), 4.23(q, J=7.2Hz, 2H), 4.73(q, J=6.9Hz, 1H), 6.90˜7.60(m, 4H); MS(70eV) m/z 219(M⁺), 146, 119, 102, 91, 73, 65 |
| 15 | 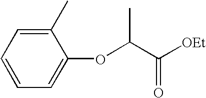 | 94.6/ 5.4 | 96% | white liquid; R_{f}=0.69(EA:Hx=1:4); ¹H NMR(CDCl₃, 200MHz): δ 1.24(t, J=7.2Hz, 3H), 1.62(d, J=6.6Hz, 3H), 2.28(s, 3H), 4.21(q, J=7.2Hz, 2H), 4.73(q, J=6.8Hz, 1H), 6.66˜7.16(m, 4H); MS(70eV) m/z 208(M⁺), 135, 108, 91, 77, 65, 55 |
| 16 | 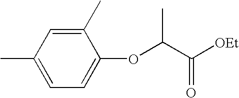 | 94.6/ 5.4 | 87% | white liquid; R_{f}=0.76(EA:Hx=1:4); ¹H NMR(CDCl₃, 200MHz): δ 1.25(t, J=7.2Hz, 3H), 1.61(d, J=6.8Hz, 3H), 2.24(s, 6H), 4.20(q, J=7.2Hz, 2H), 4.68(q, J=6.8Hz, 1H), 6.57˜6.95(m, H); MS(70eV) m/z 222(M⁺), 149, 122, 105, 91, 77 |
| 17 | 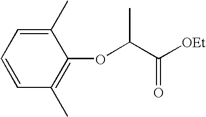 | 98.0/ 2.0 | 75% | yellow liquid; R_{f}=0.74(EA:Hx=1:4); ¹H NMR(CDCl₃, 200MHz): δ 1.28(t, J=7.2Hz, 3H), 1.53(d, J=6.6Hz, 3H), 2.29(s, 6H), 4.25(q, J=7.2Hz, 2H), 4.49(q, J=6.8Hz, 1H), 6.90˜7.02(m, 3H); MS(70eV) m/z 222(M¹), 149, 122 105, 91, 77, 65, 53 |
| 18 | 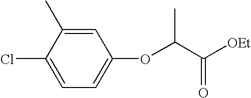 | 94.4/ 5.6 | 96% | white liquid; R_{f}=0.72(EA:Hx=1:4);¹H NMR(CDCl₃, 200MHz): δ 1.25(t, J=7.2Hz, 3H), 1.60(d, J=6.8Hz, 3H), 2.32(s, 3H), 4.22(q, J=7.2Hz, 2H), 4.69(q, J=6.8Hz, 1H), 6.61˜7.23(m, 3H); MS(70eV) m/z 244(M⁺), 242(M⁺), 169, 125, 142, 107, 99, 89 |
| 19 | 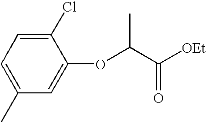 | 94.9/ 5.1 | 95% | white liquid; R_{f}=0.65(EA:Hx=1:4);¹H NMR(CDCl₃, 200MHz): δ 1.25(t, J=7.2Hz, 3H), 1.60(d, J=6.8Hz, 3H), 2.32(s, 3H), 4.22(q, J=7.2Hz, 2H), 4.69(q, J=6.8Hz, 1H), 6.60˜7.23(m, 3H); MS(70eV) m/z 244(M⁺), 242(M⁺), 169, 142, 125, 107, 99, 89 |
| 20 | 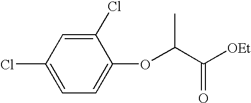 | 100.0/ 0.0 | 91% | white liquid; R_{f}=0.63(EA:Hx=1:4);¹H NMR(CDCl₃, 200MHz): δ 1.25(t, J=7.2Hz, 3H), 1.67(d, J=6.8Hz, 3H), 4.22(q, J=7.0Hz, 2H), 4.71(q, J=6.8Hz, 1H), 6.76˜7.39(m, 3H); MS(70eV) m/z 263(M⁺), 262(M+), 189, 162, 154, 145, 133, 125, 109, 101, 73 |
| 21 | 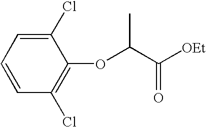 | 100.0/ 0.0 | 92% | white liquid; R_{f}=0.60(EA:Hx=1:4); ¹H NMR(CDCl₃, 200MHz): δ 1.28(t, J=7.2Hz, 3H), 1.63(d, J=6.6Hz, 3H), 4.25(q, J=7.2Hz, 2H), 4.83(q, J=7.0Hz, 1H), 6.95˜7.33(m, 3H); MS(70eV) m/z 263(M⁺), 262(M⁺), 227, 189, 162, 145, 133, 125, 109, 101, 73 |
| 22 | 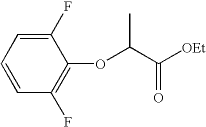 | 100.0/ 0.0 | 94% | white liquid; R_{f}=0.68(EA:Hx=1:4); ¹H NMR(CDCl₃, 200MHz): δ 1.27(t, J=7.2Hz, 3H), 1.63(d, J=6.8Hz, 3H), 4.22(q, J=7.0Hz, 2H), 4.81(q, J=7.0Hz, 1H), 6.84˜7.00(m, 3H); MS(70eV) m/z 230(M⁺), 157, 130 113, 101, 82, 73 |
| 23 | 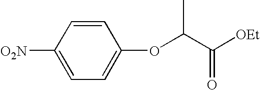 | 100.0/ 0.0 | 67% | yellow liquid; R_{f}=0.50(EA:Hx=1:2); ¹H NMR(CDCl₃, 300MHz): δ 1.26(t, J=7.2Hz, 3H), 1.68(d, J=6.6Hz, 3H), 4.24(q, J=7.1Hz, 2H), 4.85(q, J=7.2Hz, 1H), 6.90˜8.22(m, 4H); MS(70eV) m/z 239(M⁺), 166, 120 91, 76 |
| 24 | 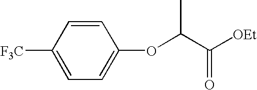 | 97.9/ 2.1 | 79% | white liquid; R_{f}=0.70(EA:Hx=1:2); ¹H NMR(CDCl₃, 300MHz): δ 1.25(t, J=7.1Hz, 3H), 1.64(d, J=6.8Hz, 3H), 4.23(q, J=7.1Hz, 2H), 4.79(q, J=6.8Hz, 1H), 6.92˜7.55(m, 4H); MS(70eV) m/z 262(M⁺), 243, 189 162, 145 |
| 25 | 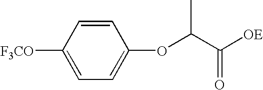 | 96.8/ 3.2 | 86% | white liquid; R_{f}0.72(EA:Hx=1:2); ¹H NMR(CDCl₃, 300MHz): δ 1.25(t, j=7.2Hz, 3H), 1.62(d, J=6.6Hz, 3H), 4.22(q, J=7.2Hz, 2H), 4.71(q, J=6.8Hz, 1H), 6.85˜7.14(m, 4H); MS(70eV) m/z 278(M⁺), 205, 178, 109, 91 |

### EXAMPLE 2

### Preparation of (D+)-ethyl-2-[4-(6-chloro-2-benzoxazolyloxy)-phenoxy]-propionate (Compound 26, Commercial Name: Fenoxaprop-p-ethyl)

50 mL of cyclohexane, 2.61 g (10 mmol) of (6-chloro-2-benzoxazolyloxy)phenol, 2.86 g (10.5 mmol) of (S)-ethyl O-p-toluenesulfonyl lactate, and 2.76 g (20 mmol) of powdery K₂CO₃ were put in a 100 mL flask equipped with a cooling condenser-attached Dean-Stock and reacted for 12 hours while refluxing. The reaction mixture was filtered without cooling and the solid cake was washed with 20 mL of warm cyclohexane. The cyclohexane layer, the filtrate, was condensed to obtain 3.20 g of the target compound (yield=89%; purity=98%; optical purity=99.9%). mp 82^{˜}84° C.(observed); Rf=0.52(hexane/ethylacetate=3/1); 1H-NMR(CDCl3, 200 MHz) δ 1.13(t, J=7.1 Hz, 3H), 1.81(d, J=6.9 Hz, 3H), 4.22(q, J=7.1 Hz, 2H), 4.72(q, J=6.9 Hz, 1H), 6.99^{˜}7.42(m, 7H); MS(70 eV) m/z 363(M+), 361(M+), 291, 288, 263, 261, 182, 144, 119, 91.

The following Table 2 shows yields and ratio of optical isomers generated in the course of substitution reactions performed the same as in Example 2.

TABLE 2

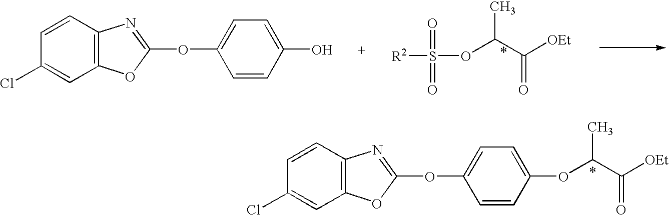

| | | | | | Ratio of |
|---|---|---|---|---|---|
| Reaction | | Reaction | Reaction | | (R)/(S) |
| Solvent | R² | Temperature | Time | Yields (g, %) | Isomers*(%) |
| Cyclohexane | p-toluyl | Reflux | 12 hours | 3.20 g, 89% | 99.9/0.1 |
| Methyl- | p-toluyl | Reflux | 12 hours | 3.20 g, 89% | 98.5/1.5 |
| cyclohexane | | | | | |
| n-Hexane | p-toluyl | Reflux | 24 hours | 2.80 g, 77.5% | 99.9/0.1 |
| Xylene | p-toluyl | 100° C. | 12 hours | 3.10 g, 85.5% | 99.9/0.1 |
| Cyclohexane | Phenyl | Reflux | 12 hours | 3.20 g, 89% | 99.9/0.1 |
| Cyclohexane | Methyl | Reflux | 12 hours | 3.20 g, 89% | 95.0/5.0 |

*Ratio of (R)/(S) isomers: Identified by LC

### EXAMPLE 3

### Preparation of (D+)-methyl-2-[4-(6-chloro-2-benzoxazolyloxy)-phenoxy]-propionate (Compound 27)

50 mL of cyclohexane, 2.61 g (10 mmol) of (6-chloro-2-benzoxazolyloxy)phenol, 2.35 g (10.5 mmol) of (S)-methyl O-(p-methoxybenzene)sulfonyl lactate, and 2.12 g (20 mmol) of powdery Na₂CO₃ were put in a 100 mL flask equipped with a cooling condenser-attached Dean-Stock and reacted for 12 hours while refluxing. The reaction mixture was filtered without cooling and the solid cake was washed with 20 mL of warm cyclohexane. The cyclohexane layer, the filtrate, was condensed to obtain 3.10 g of the target compound (yield=89%; purity=98%; optical purity=99.9%). mp 97° C.(observed); Rf=0.50(hexane/ethylacetate=3/1); 1H-NMR(CDCl3, 200 MHz) δ 1.51(d, J=6.4 Hz, 3H), 3.70(s,3H), 4.55(q, J=6.4 Hz, 1H), 6.84^{˜}7.40(m, 7H); MS(70 eV) m/z 349(M+), 347(M+), 291, 288, 263, 261, 182, 144, 119, 91.

The following Table 3 shows yields and ratio of optical isomers generated in the course of substitution reactions performed the same as in Example_{—}3.

TABLE 3

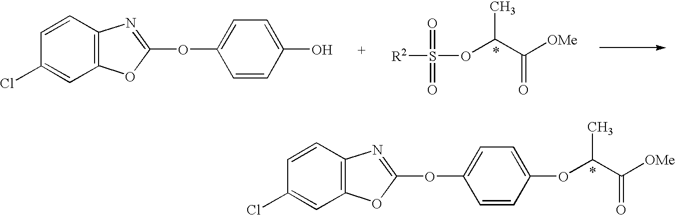

| | | | | | Ratio of |
|---|---|---|---|---|---|
| Reaction | | Reacture | Reaction | Yields | (R)/(S) |
| Solvent | R² | Temperature | Time | (g, %) | Isomers*(%) |
| Cyclohexane | p-Methoxy- | Reflux | 12 hours | 3.10 g, 89% | 99.9/0.1 |
| | phenyl | | | | |
| Methyl- | p-Methoxy- | Reflux | 12 hours | 3.10 g, 89% | 98.5/1.5 |
| cyclo- | phenyl | | | | |
| hexane | | | | | |
| n-Heptane | p-Methoxy- | Reflux | 20 hours | 2.70 g, 77.7% | 99.9/0.1 |
| | phenyl | | | | |
| Xylene | p-Methoxy- | 100° C. | 10 hours | 3.10 g, 89% | 99.9/0.1 |
| | phenyl | | | | |
| Cyclohexane | Methyl | Reflux | 12 hours | 3.05 g, 87.7% | 95.0/5.0 |
| Cyclohexane | Phenyl | Reflux | 12 hours | 3.05 g, 87.7% | 99.9/0.1 |

*Ratio of (R)/(S) isomers: Identified by LC

### EXAMPLE 4

### Preparation of (D+)-n-butyl-2-[4-(6-chloro-2-benzoxazolyloxy)-phenoxy]-propionate (Compound 28)

50 mL of cyclohexane, 2.61 g (10 mmol) of (6-chloro-2-benzoxazolyloxy)phenol, 3.15 g (10.5 mmol) of (S)-n-butyl O-p-toluenesulfonyl lactate, and 2.76 g (20 mmol) of powdery K₂CO₃ were put in a 100 mL flask equipped with a cooling condenser-attached Dean-Stock and reacted for 12 hours while refluxing. The reaction mixture was filtered without cooling and the solid cake was washed with 20 mL of warm cyclohexane. The cyclohexane layer, the filtrate, was condensed to obtain 3.60 g of the target compound (yield=92.3%; purity=98%; optical purity=99.9%). mp 48^{˜}50° C.(observed); Rf=0.59(hexane/ethylacetate=3/1); 1H-NMR(CDCl3, 200 MHz) δ 0.91(t, J=7.1 Hz, 3H), 1.48^{˜}1.58(m, 4H), 1.51(d, J=6.9 Hz, 3H), 4.26(q, J=7.1 Hz, 2H), 4.45(q, J=6.9 Hz, 1H), 6.84^{˜}7.40(m, 7H); MS(70 eV) m/z 391(M+), 389(M+), 291, 288, 263, 261, 182, 144, 119, 91.

The following Table 4 shows yields and ratio of optical isomers generated in the course of substitution reactions performed in Example 4.

TABLE 4

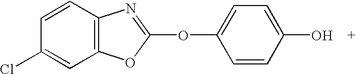

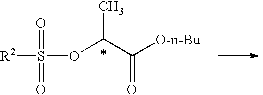

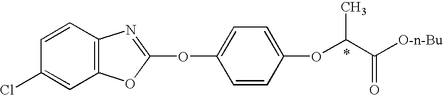

| | | | | | Ratio of |
|---|---|---|---|---|---|
| Reaction | Reaction | Reaction | Yields | (R)/(S) | |
| Solvent | R² | Temperature | Time | (g, %) | Isomers (%)* |
| Cyclohexane | p-Toluyl | Reflux | 12 hours | 3.60 g, 92.3% | 99.9/0.1 |
| Methylcyclohexane | p-Toluyl | Reflux | 12 hours | 3.60 g, 92.3% | 98.5/1.5 |
| n-Heptane | p-Toluyl | Reflux | 10 hours | 3.30 g, 84.7% | 99.9/0.1 |
| Xylene | p-Toluyl | 100° C. | 10 hours | 3.50 g, 89.8% | 99.9/0.1 |
| Xylene | p-Toluyl | 110° C. | 10 hours | 3.50 g, 89.8% | 95.0/5.0 |
| Cyclohexane | Methyl | Reflux | 12 hours | 3.50 g, 89.8% | 95.0/5.0 |
| Cyclohexane | Phenyl | Reflux | 12 hours | 3.50 g, 89.8% | 99.9/0.1 |

*Ratio of (R)/(S) isomers: Identified by LC

### EXAMPLE 5

### Preparation of (D+)-n-ethyl-2-[4-(3-chloro-5-trifluoromethylpyridine-yloxy)-phenoxy]-propionate (Compound 29)

30 mL of cyclohexane, 2.90 g (10 mmol) of 4-(3-chloro-5-trifluoromethylpyridinyloxy)phenol, 2.86 g (10.5 mmol) of (S)-ethyl O-p-toluenesulfonyl lactate, and 2.76 g (20 mmol) of powdery K₂CO₃ were put in a 50 mL flask equipped with a cooling condenser-attached Dean-Stock and reacted for 18 hours while refluxing. The reaction mixture was filtered without cooling and the solid cake was washed with 20 mL of warm cyclohexane. The cyclohexane layer, the filtrate, was condensed to obtain 3.51 g of the target compound (yield=90%; purity=98%; optical purity=97.0%).

Rf=0.56(EA:Hx=1:4); 1H NMR(CDCl3, 200 MHz) δ 1.27(t, J=7.2 Hz, 3H), 1.63(d, J=6.6 Hz, 3H), 4.24(q, J=7.2 Hz, 2H), 4.73(q, J=6.90 Hz, 1H), 6.89^{˜}8.27(m, 6H); MS(70 eV) m/z 389(M+), 370, 316, 288, 272, 261, 226, 209, 180, 160, 119, 109, 91, 76, 63.

### EXAMPLE 6

### Preparation of (D+)-n-ethyl-2-[4-(2,4-dichlorophenoxy)-phenoxy]-propionate (Compound 30)

30 mL of cyclohexane, 2.55 g (10 mmol) of 4-(2,4-dichlorophenoxy)phenol, 2.86 g (10.5 mmol) of (S)-ethyl O-p-toluenesulfonyl lactate, and 2.76 g (20 mmol) of powdery K₂CO₃ were put in a 50 mL flask equipped with a cooling condenser-attached Dean-Stock and reacted for 17 hours while refluxing. The reaction mixture was filtered without cooling and the solid cake was washed with 20 mL of warm cyclohexane. The cyclohexane layer, the filtrate, was condensed to obtain 2.74 g of the target compound (yield=77%; purity=98%; optical purity=94.6%). Rf=0.77(EA:Hx=1:2); 1H NMR(CDCl3, 300 MHz) δ 1.26(t, J=7.2 Hz, 3H), 1.62(d, J=6.9 Hz, 3H), 4.23(q, J=7.1 Hz, 2H), 4.69(q, J=6.7 Hz, 1H), 6.78^{˜}7.44(m, 7H); MS(70 eV) m/z 355(M+), 354(M+), 281, 253, 202, 184, 173, 162, 139, 120, 109, 91.

### EXAMPLE 7

### Preparation of (D+)-n-ethyl-2-[7-(2-chloro-4-trifluoromethylphenoxy)-naphthalene-2-yloxy]propionate (Compound 31))

30 mL of cyclohexane, 3.39 g (10 mmol of 7-(2-chloro-4-trifluoromethylphenoxy)-2-naphthalenol, 2.86 g (10.5 mmol) of (S)-ethyl O-p-toluenesulfonyl lactate, and 2.76 g (20 mmol) of powdery K₂CO₃ were put in a 50 mL flask equipped with a cooling condenser-attached Dean-Stock and reacted for 19 hours while refluxing. The reaction mixture was filtered without cooling and the solid cake was washed with 20 mL of warm cyclohexane. The cyclohexane layer, the filtrate, was condensed to obtain 4.08 g of the target compound (yield=93%; purity=98%; optical purity=92.8%).

Rf=0.60(EA:Hx=1:4); 1H NMR(CDCl3, 300 MHz) δ 1.24(t, J=7.2 Hz, 3H), 1.67(d, J=6.9 Hz, 3H), 4.23(q, J=5.7 Hz, 2H), 4.86(q, J=6.9 Hz, 1H), 6.94 ^{˜}7.81(m, 9H) MS(70 eV) m/z 438(M+), 365, 338, 321, 303, 286, 275, 170, 142, 126, 114, 102.

### EXAMPLE 8

### Preparation of (D+)-n-ethyl-2-[4-(6-chloroquinoxalin-2-yloxy)phenoxy]propionate (Compound 32)

30 mL of cyclohexane, 2.73g (10 mmol) of 4-(6-chloroquinoxalin-2-yloxy)phenol, 2.86 g (10.5 mmol) of (S)-ethyl O-p-toluenesulfonyl lactate, and 2.76 g (20 mmol) of powdery K₂CO₃ were put in a 50 mL flask equipped with a cooling condenser-attached Dean-Stock and reacted for 18 hours while refluxing. The reaction mixture was filtered without cooling and the solid cake was washed with 20 mL of warm cyclohexane. The cyclohexane layer, the filtrate, was condensed to obtain 3.39 g of the target compound (yield=91%; purity=98%; optical purity=99.8%).

mp=60^{˜}61° C.(R observed), mp=83^{˜}84° C.(R,S observed), Rf=0.63(EA:Hx=1:2); 1H NMR(CDCl3, 500 MHz) δ 1.29(t, J=7.1 Hz, 3H), 1.65(d, J=6.8 Hz, 3H), 4.26(m, 2H), 4.76(q, J=6.8 Hz, 1H), 6.95^{˜}8.67(m, 7H); MS(70 eV) m/z 372(M+), 299, 272, 255, 244, 212, 199, 163, 155, 136, 110, 100, 91, 65.

The following Table 1 shows the yield, ratio of generated optical isomers and spectral data of the compounds (33-38) performed in Example 8.

TABLE 5

| comp. | | R/S | | |
|---|---|---|---|---|
| no. | structure | ratio | yields | mp, R_{f},NMR, MS |
| 33 | 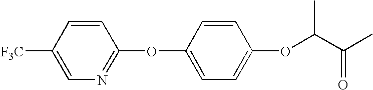 | 99.3/ 0.7 | 92% | white solid, mp=33˜35° C.; R_{f}=0.58(EA:Hx=1:4); ¹H NMR(CDCl₃, 200MHz): δ1.28(t, 1=7.2Hz, 3H), 1.63(d, J=6.8Hz, 3H), 4.24(q, J=7.1Hz 2H), 4.73(q, J=6.8Hz, 1H), 6.94˜8.44(m, 7H); MS(70eV) m/z 355(M⁺), 336, 282, 254, 227, 198, 146, 126, 91, 76 |
| 34 | 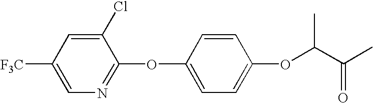 | 96.9/ 3.1 | 94% | yellow liquid; R_{f}=0.75(EA:Hx=1:2); ¹H NMR(CDCl₃, 200MHz): δ1.27(t, J=7.2Hz, 3H), 1.63(d, J=6.4Hz, 3H), 4.24(q, J=7.1Hz, 2H), 4.72(q, J=6.8Hz, 1H), 6.83˜7.71(m, 7H); MS(70eV) m/z 388(M⁺), 369, 315, 288, 253, 236, 196, 179, 157, 120, 109, 91, 64 |
| 35 | 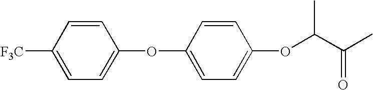 | 97.0/ 3.0 | 96% | white solid, mp=58˜60° C. R_{f}=0.64(EA:Hx=1:4); ¹H NMR(CDCl₃, 200MHz): δ1.27(t, J=7.2Hz, 3H), 1.63(d, J=6.6Hz, 3H), 4.24(q, J=7.1Hz, 2H), 4.72(q, J=6.8Hz, 1H), 6.87˜7.56(m, 8H); MS(70eV) m/z 354(M⁺), 335, 281, 254, 209, 177, 168, 145, 120, 109 |
| 36 | 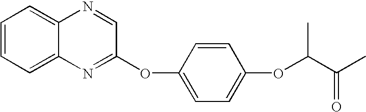 | 96.8/ 4.0 | 85% | white solid, mp=62˜65 °C.; R_{f}=0.33(EA:Hx=1:4); ¹H NMR(CDCl₃, 200MHz): δ1.28(t, J=7.2Hz, 3H), 1.65(d, J=6.8Hz, 3H), 4.25(q, J=7.1Hz, 2H), 4.77(q, J=6.8Hz, 1H), 6.91˜8.07(m, 9H); MS(70eV) m/z 338(M⁺), 310, 265, 237, 221 155, 129, 102, 91, 75 |
| 37 | 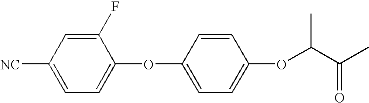 | 99.9/ 0.1 | 90% | white liquid; R_{f}=0.54(EA:Hx=1:2); ¹H NMR(CDCl₃, 200MHz): δ1.27(t, J=7.2Hz, 3H), 1.64(d, J=6.8Hz, 3H), 4.24(q, J=7.2Hz, 2H), 4.72(q, J=6.8Hz, 1H), 6.80˜7.51(m, 7H); MS(70eV) m/z 329(M⁺), 310, 272, 256, 237, 229, 199, 184, 155, 120, 101, 91 |
| 38 | 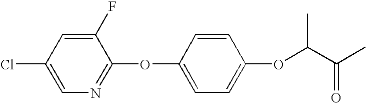 | 99.1/ 09 | 92% | white solid, mp48˜50°C.; R_{f}=0.58(EA:Hx=1:4); ¹H NMR(CDCl₃, 200MHz): δ1.28(t, J=7.2Hz, 3H), 1.63(d, J=6.8Hz, 3H), 4.24(q, J=7.1Hz 2H), 4.73(q, J=6.8Hz, 1H), 6.94˜8.44(m, 7H); MS(70eV) m/z 340(M⁺), 267, 239, 212, 183, 131, 111, 91 |

### COMPARATIVE EXAMPLE 1

The following Tables 6 and 7 show yields and ratio of optical isomers generated in the course of preparing (D+)-methyl-2-[4-(6-chloro-2-benzoxazolyloxy)phenoxy]propionate (compound 27) according to the known methods shown in the reaction schemes 1 and 2.

TABLE 7

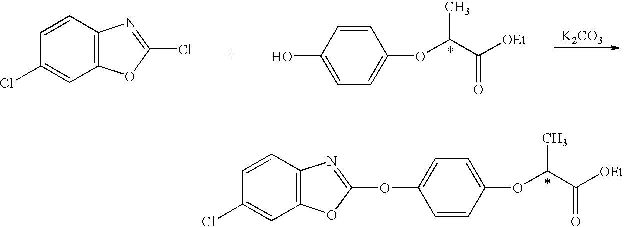

| | | | | | Ratio of |
|---|---|---|---|---|---|
| Reaction | Reaction | Reaction | | Yields | (R)/(S) |
| Solvent | Temperature | Time | | (%) | Isomers (%)* |
| Acetonitrile | Reflux | 5 | hours | 80% | 85.0/15.0 |
| Methyl ethyl | Reflux | 5 | hours | 75% | 80.0/20.0 |
| ketone | | | | | |
| Acetone | Reflux | 15 | hours | 79% | 80.0/20.0 |
| Dimethylform- | Reflux | 4 | hours | 84% | 75.0/25.0 |
| amide | | | | | |
| Dichloro- | Reflux | 15 | hours | 64% | 90.0/10.0 |
| methane | | | | | |

*Ratio of (R)/(S) isomers: Identified by LC

TABLE 7

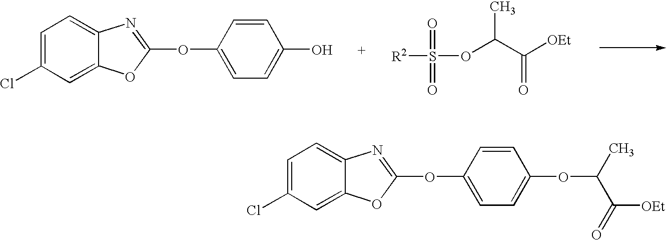

| | | | | | Ratio of |
|---|---|---|---|---|---|
| Reaction | | Reaction | Reaction | Yields | (R)/(S) |
| Solvent | R² | Temperature | Time | (%) | Isomers (%)* |
| Acetonitrile | p-Toluyl | Reflux | 5 hours | 85% | 95.0/5.0 |
| Methyl ethyl | p-Toluyl | Reflux | 5 hours | 82% | 95.0/5.0 |
| ketone | | | | | |
| Acetonitrile | Methyl | Reflux | 5 hours | 87% | 85.0/15.0 |
| Methyl ethyl | Methyl | Reflux | 5 hours | 85% | 85.0/15.0 |
| ketone | | | | | |

*Ratio of (R)/(S) isomers: Identified by LC

### COMPARATIVE EXAMPLE 2

The following Table 8 shows yields and ratio of optical isomers generated in the course of preparing (D+)-n-ethyl-2-[4-(3-chloro-5-trifluoromthylpyridine-2-yloxy)phenoxy]propionate (compound 29) according to the known methods shown in the reaction scheme 2.

TABLE 8

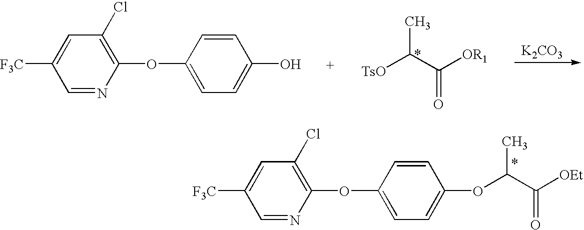

| | | | | Ratio of |
|---|---|---|---|---|
| Reaction | Reaction | Reaction | Yield | (R)/(S) |
| Solvent | Temperature | Time | (%) | Isomers (%)* |
| Acetonitrile | Reflux | 5 hours | 72% | 95.0/5.0 |
| Methyl ethyl | Reflux | 5 hours | 79% | 80/20.0 |
| ketone | | | | |
| Dimethyl- | 80˜90° C. | 4 hours | 70% | 93.0/7.0 |
| formamide | | | | |

*Ratio of (R)/(S) isomers: Identified by LC

### COMPARATIVE EXAMPLE 3

The following Table 9 shows yields and ratio of optical isomers generated in the course of preparing (D+)-n-ethyl-2-[4-(6-chloroquinoxalin-2-yloxy)phenoxy]propionate (compound 32) according to the known methods shown in the reaction scheme 2.

TABLE 9

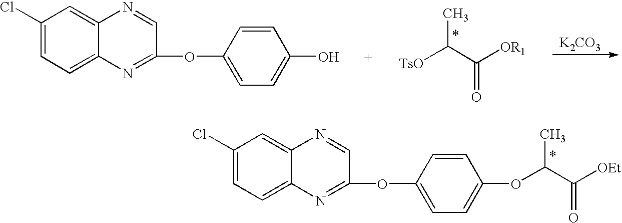

| | | | | Ratio of |
|---|---|---|---|---|
| Reaction | Reaction | Reaction | Yields | (R)/(S) |
| Solvent | Temperature | Time | (%) | Isomers (%)* |
| Acetonitrile | Reflux | 5 hours | 66% | 95.0/5.0 |
| Methyl ethyl | Reflux | 5 hours | 59% | 95.0/5.0 |
| ketone | | | | |
| Dimethyl- | 80 ˜ 90° C. | 4 hours | 63% | 93.0/7.0 |
| formamide | | | | |

*Ratio of (R)/(S) isomers: Identified by LC

### INDUSTRIAL APPLICABILITY

As described above, the preparing method of the present invention enables production of optically pure (R)-aryloxy propionic acid ester derivatives with good yield and is thus expected to produce an enormous economic effect.

While the present invention has been described in detail with reference to the preferred embodiments, those skilled in the art will appreciate that various modifications and substitutions can be made thereto without departing from the spirit and scope of the present invention as set forth in the appended claims.

1. A method for preparing optically active (R)-aryloxypropionic acid ester derivatives represented by the following Formula 1 by reacting phenol derivatives represented by the following Formula 2 and (S)-alkyl O-arylsulfonyl lactate represented by the following Formula 3 in the presence of alkali metal carbonate in an aliphatic or aromatic hydrocarbon solvent under the temperature range of 60 to 100° C.: 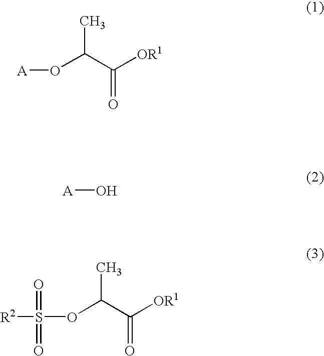
wherein water formed during the reaction is continuously removed, and
wherein R¹ is a C₁₋₆-alkyl or benzyl group; R² is a C₁₋₆-alkyl, phenyl group, or a phenyl group substituted with a C₁₋₆-alkyl or a C₁₋₆-alkoxy group; A is an aryl group selected from the group consisting of a phenyl group, a naphthyl group, a quinoxazolyloxyphenly group, a benzoxazolyloxyphenyl group, a benzothiazolyloxyphenyl group, a phenyloxyphenyl group, a pyridyloxyphenyl group and a pheyloxynaphthyl group, wherein said aryl group can be substituted with 1-3 functional groups selected from the group consisting of a halogen atom, a nitro group, a nitrile group, an acetoxy group, a C₁₋₄-alkyl group, a C₁₋₄-haloalkyl group, a C₁₋₄-alkoxy group, and a C₁₋₄-haloalkoxy group.

2. In claim 1, said hydrocarbon solvent is selected from the group consisting of toluene, xylene, cyclopentane, cyclohexane, methylcyclohexane, cycloheptane, n-hexane, and n-heptane.

3. In claim 1, said solvent is cyclohexane or xylene.

4. In claim 1, said method for preparing optically active (R)-aryloxypropionic acid ester derivatives is performed using potassium carbonate as a base in cyclohexane as a solvent at 80° C.

5. In claim 1, the water is removed by using a flask equipped with a cooling condenser and Dean-Stock.
